(19) United States
(12) Patent Application Publication
(10) Pub. No.: US 2025/0268799 A1
(43) Pub. Date: Aug. 28, 2025

(54) MEDIUM FOR WATER-FREE ROOT CANAL DISINFECTANT WITH HIGH FLUIDITY, DISINFECTANT USING SAME, AND USE THEREOF

(71) Applicant: WENZHOU QINGCHENG HEALTHCARE TECH LTD., Wenzhou, Zhejiang (CN)

(72) Inventors: Lei ZHANG, Wenzhou (CN); Yu SUN, Wenzhou (CN); Kewang LU, Wenzhou (CN); Tuojiang WU, Wenzhou (CN); Yang SONG, Wenzhou (CN); Xiaojun SHI, Wenzhou (CN)

(21) Appl. No.: 18/858,575

(22) PCT Filed: Jun. 24, 2022
(86) PCT No.: PCT/CN2022/101161

§ 371 Date: Oct. 21, 2024

(30) Foreign Application Priority Data
Apr. 21, 2022 (CN) 202210420813.0

(51) Int. Cl. A61K 6/52; A61K 6/69; A61K 6/70

(52) U.S. Cl. CPC A61K 6/52; A61K 6/69; A61K 6/70

(57) ABSTRACT

A matrix of an anhydrous root canal disinfectant with high fluidity, the disinfectant and application thereof. The matrix comprises 0.5-5.0% polyvinylpyrrolidone, 0.1-5.0% nonionic or cationic surfactant, and 32.0-85.0% polyethylene glycol with a molecular weight of 200-600 based on the total mass percentage of the anhydrous root canal disinfectant.

### CROSS-REFERENCE TO RELATED APPLICATIONS

This application is an U.S. national phase application under 35 U.S.C. § 371 based upon international patent application No. PCT/CN2022/101161 filed on Jun. 24, 2022, which itself claims priority to Chinese patent application No. 202210420813.0, filed on Apr. 21, 2022. The contents of the above identified applications are hereby incorporated herein in their entireties by reference.

### TECHNICAL FIELD

The present disclosure relates to the field of oral care technology, and in particular, to a highly flowable anhydrous root canal disinfectant matrix, and a disinfectant and applications thereof using the same.

### BACKGROUND

Root canal therapy, also known as endodontic treatment, is a dental procedure used to treat necrotic dental pulp and root infections. The primary objectives of this treatment are to eliminate bacteria, remove biofilms, seal the root canal to prevent reinfection, and promote the healing of periapical tissues. Root canal therapy consists of three stages: canal preparation, disinfection, and filling. Due to the complexity of the root canal system, conventional mechanical preparation can only remove most of the infected tissue, failing to achieve complete debridement. Bacteria and toxins that remain in the deeper dentin tubules and lateral canals are difficult to eliminate solely with manual instruments or rotary nickel-titanium files. Therefore, effective disinfection of the root canal is crucial, necessitating a disinfectant that can penetrate and sterilize the canal thoroughly.

Calcium hydroxide is the most commonly used intracanal medicament. First introduced by Hermann in 1920, it has become a widely used standard intracanal medicament. Calcium hydroxide serves as a slow-acting disinfectant in root canals, primarily through the dissociation of Ca²⁺ and OH⁻ ions, creating an alkaline environment that inhibits and kills bacteria. The differences in the physical and chemical properties of various formulations affect the release rate of Ca²⁺ and OH⁻ ions, the ability of OH⁻ ions to penetrate dentin tubules, and the duration of ion release, thereby influencing the antibacterial efficacy of calcium hydroxide paste. In vitro direct contact experiments show that complete eradication of Enterococcus faecalis requires 24 hours of contact time. Using calcium hydroxide as an intracanal medicament for one week can reduce bacteria by 92.5%, which is significantly more effective than using NaClO for root canal disinfection. Thus, using calcium hydroxide for root canal disinfection can provide more reliable, stable, and long-lasting antibacterial effects. In addition to killing bacteria, calcium hydroxide can hydrolyze the lipid component of bacterial lipopolysaccharides, thereby neutralizing their biological activity and reducing their effects. Bacterial cell walls can persist after bacteria are killed and continue to stimulate periapical tissues. Calcium hydroxide not only hydrolyzes the lipid component of bacterial lipopolysaccharides but also destroys endotoxins, thus reducing inflammatory responses in the periapical tissues.

Calcium hydroxide has multiple pharmacological actions and good bactericidal properties. It can induce apical closure, eliminate microorganisms in infected root canals, reduce inflammation within the canal, and has low toxicity to periapical tissues. Additionally, it promotes the differentiation of periapical connective tissues, enhances alkaline phosphatase activity, and facilitates the deposition of cementum-like and bone-like tissues along the canal walls, contributing to the extension of the root and closure of the apical foramen. Therefore, calcium hydroxide is currently the most favored root canal disinfectant.

However, there are several issues associated with the clinical use of calcium hydroxide: (1) The two-component calcium hydroxide pastes commonly used in clinical practice are not easy for doctors to mix; (2) It is not easy to disperse, making it difficult to completely remove or rinse out before canal filling; (3) It tends to absorb carbon dioxide, forming calcium carbonate, which is ineffective for root canal treatment and difficult to remove; (4) Most clinical calcium hydroxide pastes require water for mixing, which can quickly create a high pH environment in the root canal, but some products are not able for maintaining a high pH environment over a long period.

### SUMMARY

The objective of the present disclosure is to provide a highly flowable anhydrous root canal disinfectant matrix, its application in disinfectants, and its use. The present disclosure involves the composition of the matrix using polyvinylpyrrolidone, non-ionic or cationic surfactants, and polyethylene glycol of a specific molecular weight. Then, adding an alkaline earth metal hydroxide creates a highly flowable anhydrous root canal disinfectant paste. This disinfectant paste disperses easily in water and can be quickly flushed out before root canal filling. Meanwhile, the disinfectant paste can effectively control the diffusion of OH⁻ over a long period, preventing the combination of calcium hydroxide with CO₂ produced by the decomposition of root canal environments or tissues, thereby maintaining a prolonged strongly alkaline environment in the root canal. This ensures long-term antimicrobial effects within the root canal, overcoming the shortcomings present in the current technology.

The technical solution adopted by the present disclosure is as follows: a highly flowable anhydrous root canal disinfectant matrix, calculated as a percentage of the total mass of the anhydrous root canal disinfectant, comprising the following components: 0.5-5.0% polyvinylpyrrolidone, 0.1-5.0% non-ionic or cationic surfactants, and 32.0-85.0% polyethylene glycol with a molecular weight of 200-600.

In some embodiments of the present disclosure, the mass fraction of polyvinylpyrrolidone (PVP) can be 0.5%, 0.6%, 0.7%, 0.9%, 1.0%, 1.2%, 1.3%, 1.5%, 1.6%, 1.9%, 2.0%, 2.5%, 3.0%, 3.5%, 3.6%, 3.7%, 5.0%, etc., selected according to actual needs. Furthermore, the mass fraction of the non-ionic or cationic surfactants can be 0.1%, 0.2%, 0.25%, 0.3%, 0.5%, 0.6%, 0.7%, 0.8%, 1.0%, 1.1%, 1.2%, 1.3%, 1.5%, 1.6%, 1.9%, 2.0%, 2.5%, 3.0%, 3.5%, 3.6%, 3.7%, 4.0%, 4.3%, 4.6%, 5.0%, etc., selected according to actual needs. Furthermore, the polyethylene glycol requires a molecular weight of 200-600, which is liquid at room temperature. The specific amount of polyethylene glycol, mainly serving as a solvent, generally ranges from 32.0-85.0%, adjusted appropriately during preparation.

Additionally, an alkaline earth metal hydroxide is added to the matrix to prepare the paste, which is the anhydrous root canal disinfectant.

Optionally, the alkaline earth metal hydroxide is calcium hydroxide.

Optionally, the non-ionic surfactant is polyoxyethylene ether hydrogenated castor oil, and the cationic surfactant is chlorhexidine.

Furthermore, the present disclosure also includes a highly flowable anhydrous root canal disinfectant, which includes the aforementioned anhydrous root canal disinfectant matrix and an alkaline earth metal hydroxide. The amount of alkaline earth metal hydroxide is 10-60% of the total mass of the anhydrous root canal disinfectant. The amount of alkaline earth metal hydroxide can be selected based on actual conditions, such as 10%, 11%, 15%, 16%, 18%, 19%, 20%, 21%, 23%, 25%, 27%, 29%, 30%, 32%, 35%, 36%, 38%, 40%, 42%, 45%, 47%, 50%, 52%, 55%, 56%, 58%, 60%, etc. Generally, the fewer other additives in the anhydrous root canal disinfectant, the higher the amount of alkaline earth metal hydroxide. For instance, when no X-ray contrast agent is added to the anhydrous disinfectant, the amount of alkaline earth metal hydroxide can reach over 35%, even up to 60%.

Furthermore, the anhydrous root canal disinfectant also contains an X-ray contrast agent, optionally zirconium oxide.

Additionally, the anhydrous root canal disinfectant contains an antibacterial agent, which can be single-chain or double-chain cationic quaternary ammonium salts and/or chlorhexidine.

Furthermore, calculated as a percentage of the total mass of the anhydrous root canal disinfectant, the anhydrous root canal disinfectant includes the following components: 0.5-5.0% polyvinylpyrrolidone, 0.1-5.0% polyoxyethylene ether hydrogenated castor oil, 10-60% calcium hydroxide, 15-35% zirconium oxide, with the balance being polyethylene glycol with a molecular weight of 200-600 and other additives.

Additionally, the present disclosure also includes a method for preparing a highly flowable anhydrous root canal disinfectant, comprising the following steps:
Step 1. Place polyvinylpyrrolidone into polyethylene glycol and allow it to swell fully to obtain a homogeneous solution A;
Step 2. Add the remaining components to the homogeneous solution A, homogenize and disperse to obtain the final product.

Furthermore, the present disclosure includes the application of the highly flowable anhydrous root canal disinfectant in root canal treatment. The anhydrous root canal disinfectant is prepared using the aforementioned preparation method. In root canal treatment, the anhydrous root canal disinfectant is injected into the deep parts of the root canal using a 25 G root canal irrigation needle for disinfection, followed by irrigation with water or root canal irrigation solution.

In summary, the beneficial effects of the embodiments of the present disclosure, which are not intended to limit the protection scope of the present disclosure, are as follows:

The anhydrous root canal disinfectant of this disclosure can be used directly without the need for preparation before use, avoiding the inconvenience.

The anhydrous root canal disinfectant of this disclosure disperses easily in water and can be flushed out easily before root canal filling, avoiding the issues in existing products of poor dispersion and difficulty in flushing out.

The anhydrous root canal disinfectant of the present disclosure can effectively control the diffusion of OH⁻ over a long period, preventing the combination of calcium hydroxide with CO₂ produced by the environment or tissue decomposition, thereby maintaining a strongly alkaline environment in the root canal and ensuring long-term antimicrobial effects within the root canal.

The anhydrous root canal disinfectant of this disclosure can penetrate dentinal tubules, achieving deep disinfection and remineralization, with excellent disinfecting, antibacterial, and desensitizing effects.

The anhydrous root canal disinfectant of the present disclosure has high radiopacity, with an X-ray opacity value ≥300% Al.

### BRIEF DESCRIPTION OF THE DRAWINGS

FIGS. 1A to 1D shows the dispersion test results of paste A in the water dispersion experiment.

FIGS. 2A to 2D shows the dispersion test results of paste B in the water dispersion experiment.

FIGS. 3A to 3D shows the dispersion test results of paste C in the water dispersion experiment.

FIGS. 4A to 4D shows the dispersion test results of paste D in the water dispersion experiment.

FIGS. 5A to 5D shows the dispersion test results of paste E in the water dispersion experiment.

FIGS. 6A to 6D shows the dispersion test results of paste F in the water dispersion experiment.

FIGS. 7A to 7D shows the dispersion test results of paste G in the water dispersion experiment.

FIGS. 8A to 8D shows the dispersion test results of paste H in the water dispersion experiment.

FIGS. 9A to 9D shows the dispersion test results of paste A1 in the water dispersion experiment.

FIGS. 10A to 10D shows the dispersion test results of paste A2 in the water dispersion experiment.

FIGS. 11A to 11D shows the dispersion test results of paste A3 in the water dispersion experiment.

FIGS. 12A to 12D shows the dispersion test results of paste A4 in the water dispersion experiment.

FIGS. 13A to 13D shows the dispersion test results of paste A5 in the water dispersion experiment.

FIGS. 14A to 14D shows the dispersion test results of paste A6 in the water dispersion experiment.

FIGS. 15A to 15D shows the dispersion test results of paste A7 in the water dispersion experiment.

FIGS. 16A to 16D shows the dispersion test results of paste A8 in the water dispersion experiment.

FIGS. 17A to 17D shows the dispersion test results of paste A9 in the water dispersion experiment.

FIGS. 18A to 18D shows the dispersion test results of paste A10 in the water dispersion experiment.

FIGS. 19A to 19D shows the dispersion test results of paste A11 in the water dispersion experiment.

FIGS. 20A to 20D shows the dispersion test results of paste A12 in the water dispersion experiment.

FIGS. 21A to 21D shows the dispersion test results of paste A13 in the water dispersion experiment.

FIGS. 22A to 22D shows the dispersion test results of paste A14 in the water dispersion experiment.

FIGS. 23A to 23D shows the dispersion test results of paste A15 in the water dispersion experiment.

FIGS. 24A to 24C shows the flowability comparison experiment between Formula 3 paste and commercially available products LQC and APC, where FIG. 24A is the compression film of Formula 3 paste, FIG. 24B is the compression film of LQC, and FIG. 24C is the compression film of APC.

In FIGS. 1A-23D, numbers 1-4 (A to D) indicate the sequential dispersion of the paste in water.

FIGS. 25A to 25B shows the comparison of the status before and after rinsing the root canal of a 3D printed tooth with water, where FIG. 25A shows the status before rinsing, and FIG. 25B shows the status after rinsing.

### DETAILED DESCRIPTION

The present disclosure will be described in detail below in conjunction with the accompanying drawings.

To make the objectives, technical solutions, and advantages of the present disclosure clearer, the following provides a more detailed description of the present disclosure with reference to the accompanying drawings and exemplary embodiments. It should be understood that the specific embodiments described here are merely for explaining the present disclosure and are not intended to limit the scope of the present disclosure.

### I. The Effect of Different Solvents on CO₂ Isolation

### 1. Paste Preparation

Paste A: 25% Calcium Hydroxide, 27% Zirconium Oxide, 0.5% Polyoxyethylene (40) Hydrogenated Castor Oil, 0.5% Polyvinylpyrrolidone, and the remainder being Polyethylene Glycol 200.

Paste B: 25% Calcium Hydroxide, 27% Zirconium Oxide, 0.5% Polyoxyethylene (40) Hydrogenated Castor Oil, 0.5% Polyvinylpyrrolidone, and the remainder being Propylene Glycol.

Paste C: 25% Calcium Hydroxide, 27% Zirconium Oxide, 0.5% Polyoxyethylene (40) Hydrogenated Castor Oil, 0.5% Polyvinylpyrrolidone, and the remainder being Glycerol.

Paste D: 25% Calcium Hydroxide, 27% Zirconium Oxide, 0.5% Polyoxyethylene (40) Hydrogenated Castor Oil, 0.5% Polyvinylpyrrolidone, and the remainder being Water.

The preparation of pastes A, B, C, and D follows the method described in “Example 1”, with the variation of replacing Polyethylene Glycol 200 with Propylene Glycol, Glycerol, and Water respectively.

### 2. CO₂ Preparation

CO₂ gas is generated by mixing citric acid and sodium bicarbonate in a 1:1 ratio in a CO₂ generator.

### 3. Experimental Method

S1: Take 5 g of each of the prepared pastes and place them in 20 mL sealable transparent glass sample bottles, with four bottles for each paste.

S2: Introduce the generated CO₂ into the transparent glass bottles for 30 seconds each, sealing the bottles tightly afterward. Repeat the CO₂ introduction every hour for a total of three times.

S3: After 24 hours, carefully place 1 g and 2 g weights into each bottle, observing if the weights sink into the paste. Each weight is used twice for each bottle.

### 4. Experimental Results

Result 1: The 1 g weights remain on the surface of pastes A, B, C, and D.

Result 2: The 2 g weight sinks into paste A but remains on the surface for pastes B, C, and D.

Result 3: During the preparation of paste C, the paste quickly thickened and generated heat and an odor, differing significantly from the other three pastes. To thin the paste, twice the amount of Glycerol was added, resulting in the actual composition: 13.2% Calcium Hydroxide, 19.9% Zirconium Oxide, 0.3% Polyoxyethylene (40) Hydrogenated Castor Oil, 0.3% Polyvinylpyrrolidone, with the remainder being Glycerol.

Result 4: Upon introducing CO₂ into paste D after preparation, a hard crust quickly formed on the surface.

### 5. Experimental Conclusion

Conclusion 1: Based on the weight-bearing test and observed phenomena, the effectiveness of the four liquid solvents in isolating CO₂, from strongest to weakest, is: Polyethylene Glycol>Propylene Glycol>Water>Glycerol.

Conclusion 2: Polyethylene Glycol is more effective in preventing the reaction between CO₂ and Calcium Hydroxide to form Calcium Carbonate, while Glycerol is not suitable for use in Calcium Hydroxide pastes.

### II. Water Dispersion Test

### 1. Preparation of Pastes

Paste A: 20% Calcium Hydroxide, 30% Zirconium Oxide, 0.5% Polyvinylpyrrolidone, the rest is Polyethylene Glycol 200.

Paste B: 20% Calcium Hydroxide, 30% Zirconium Oxide, 0.5% Polyvinylpyrrolidone, the rest is Propylene Glycol.

Paste C: 20% Calcium Hydroxide, 30% Zirconium Oxide, 0.5% Polyvinylpyrrolidone, the rest is Water.

Paste D: 20% Calcium Hydroxide, 30% Zirconium Oxide, 0.5% Hydroxypropyl Methylcellulose, the rest is Polyethylene Glycol 200.

Paste E: 25% Calcium Hydroxide, 27% Zirconium Oxide, 0.5% Polyvinylpyrrolidone, 0.5% Polyoxyethylene (60) Hydrogenated Castor Oil, the rest is Polyethylene Glycol 200.

Paste F: 25% Calcium Hydroxide, 27% Zirconium Oxide, 0.5% Polyvinylpyrrolidone, 0.5% Polyoxyethylene (60) Hydrogenated Castor Oil, the rest is Propylene Glycol.

Paste G: Commercial Domestic Calcium Hydroxide Paste (LQC).

Paste H: Commercial Imported Calcium Hydroxide Paste (APC).

Preparation of Pastes A, B, C: Refer to the method described in “Example 1”, but without adding Polyoxyethylene (60) Hydrogenated Castor Oil.

Using Paste A as the base, add 0.5% (by mass) of the following types of surfactants to obtain the pastes shown in Table 1:

TABLE 1
Paste Formulations and Codes

| Type | Formulation | | Code |
|---|---|---|---|
| Cationic | Polyhexamethylene Biguanide Hydrochloride | A | A1 |
| Surfactants | (PHMB) | | |
| | Polyhexamethylene Guanidine Hydrochloride | A | A2 |
| | (PHMG) | | |
| | Benzalkonium Chloride (BZK) | A | A3 |
| | Didecyldimethylammonium Chloride (DDAC) | A | A4 |
| | Chlorhexidine Acetate (CHX) | A | A5 |
| | Dodecyltrimethylammonium Chloride (1231) | A | A6 |
| Anionic | Sodium Myristoyl Glutamate (SMG) | A | A7 |
| Surfactants | Sodium Dodecyl Sulfate (SDS) | A | A8 |
| Nonionic | Decyl Glucoside (APG-10) | A | A9 |
| Surfactants | Fatty Alcohol Polyoxyethylene Ether (AEO-9) | A | A10 |
| | Tween-80 (TW-80) | A | A11 |
| | Poloxamer407 (F127) | A | A12 |
| | Polyoxyethylene (60) Hydrogenated Castor Oil | A | A13 |
| | (RH60) | | |
| Amphoteric | Cocamidopropylamine Oxide (OA12/14) | A | A14 |
| Surfactants | Dodecyl Dimethyl Betaine (BS-12) | A | A15 |

Add appropriate food-grade carmine dye to the above pastes, homogenize thoroughly with a homogenizer so that each paste can be squeezed out from a 25 G root canal irrigation needle, and divide into syringes for use. Commercial domestic made calcium hydroxide paste (LQC) and imported calcium hydroxide paste (APC) are used directly without coloring, as they cannot be squeezed out from a 25 G root canal irrigation needle, so a regular butterfly irrigation needle is used instead.

### 2. Test Method

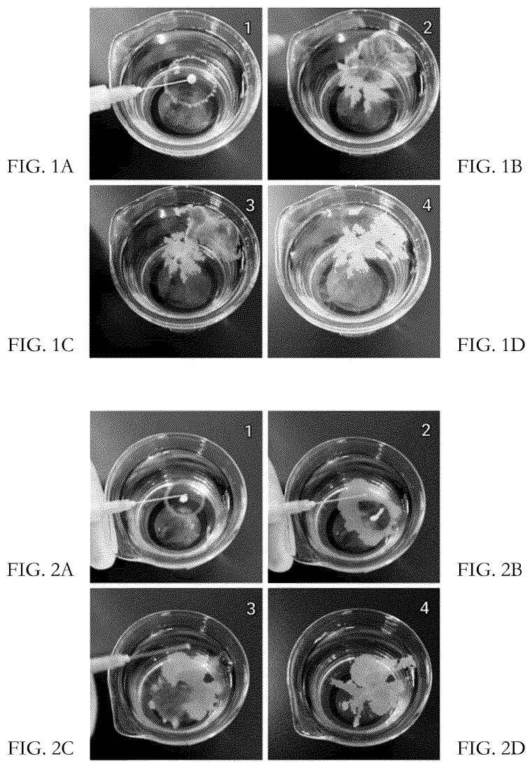

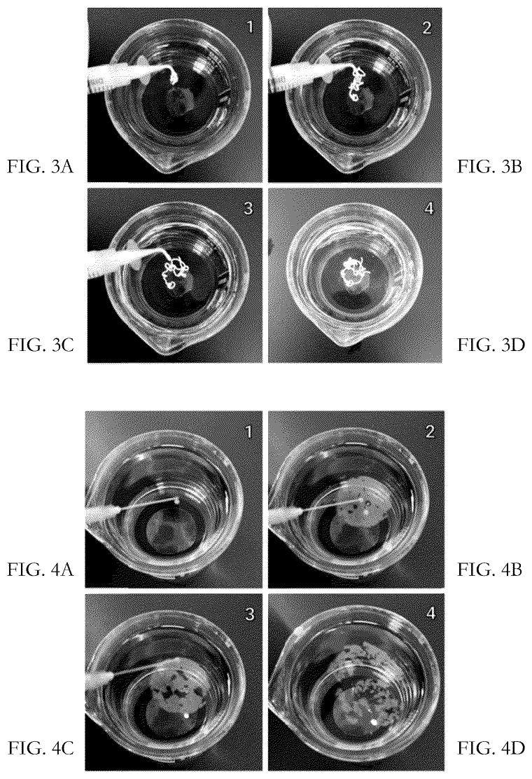

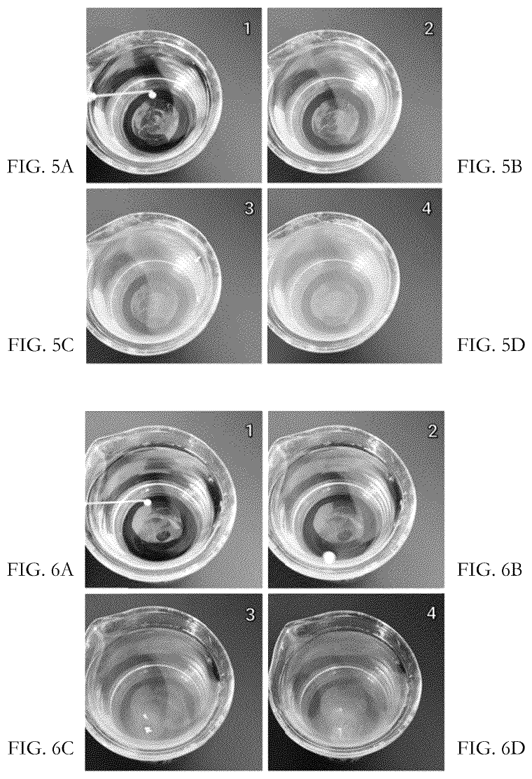

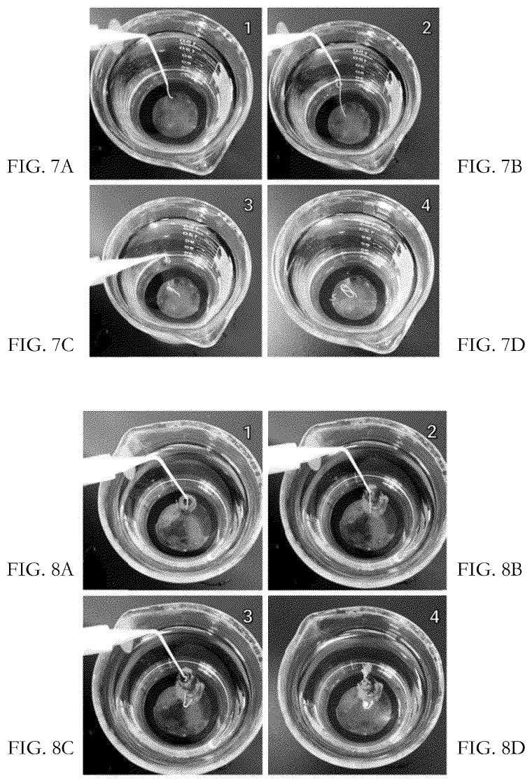

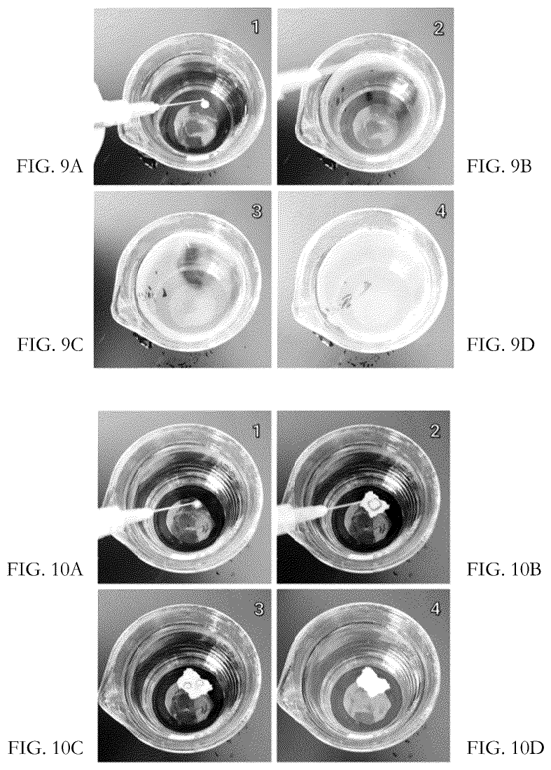

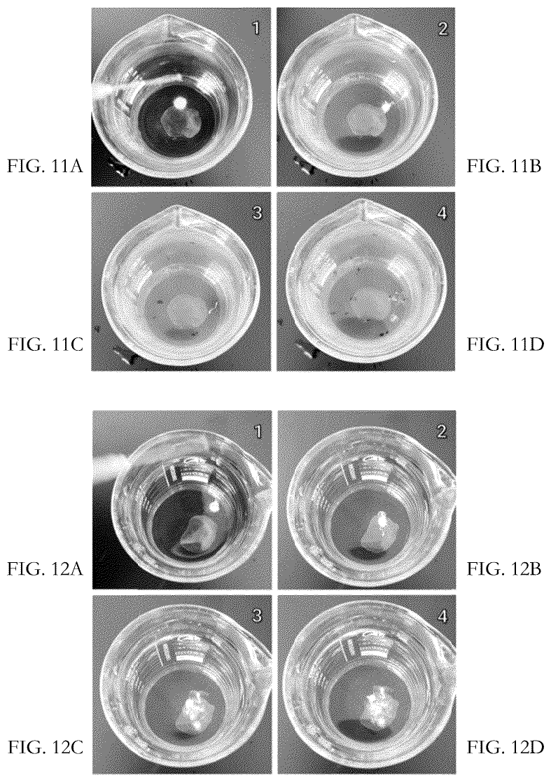

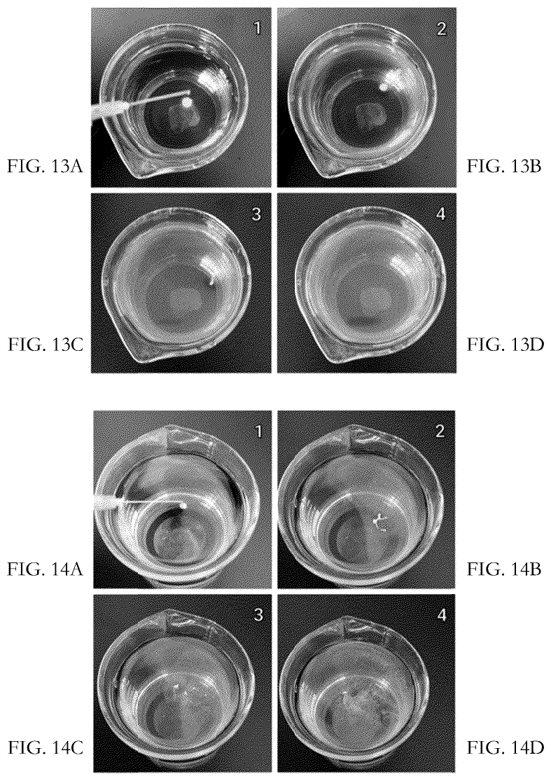

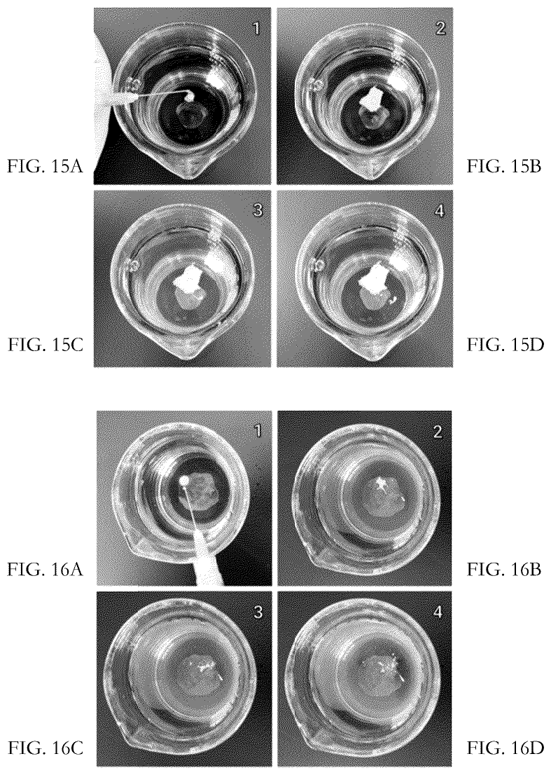

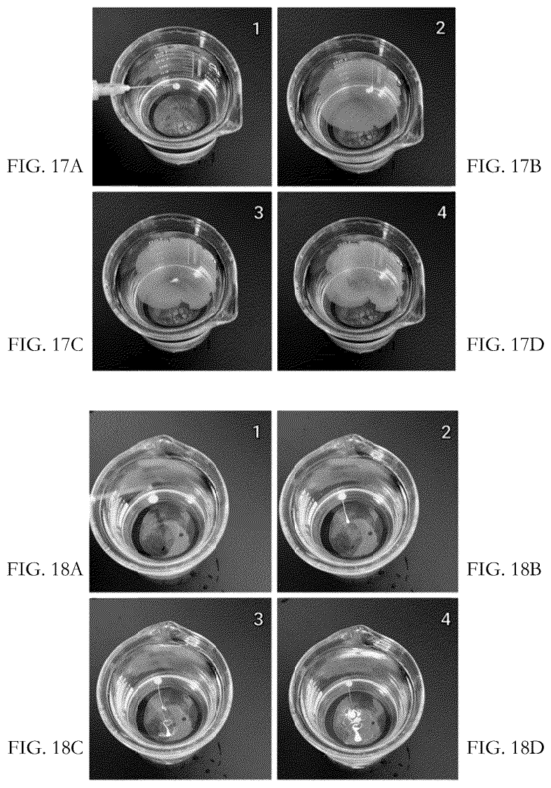

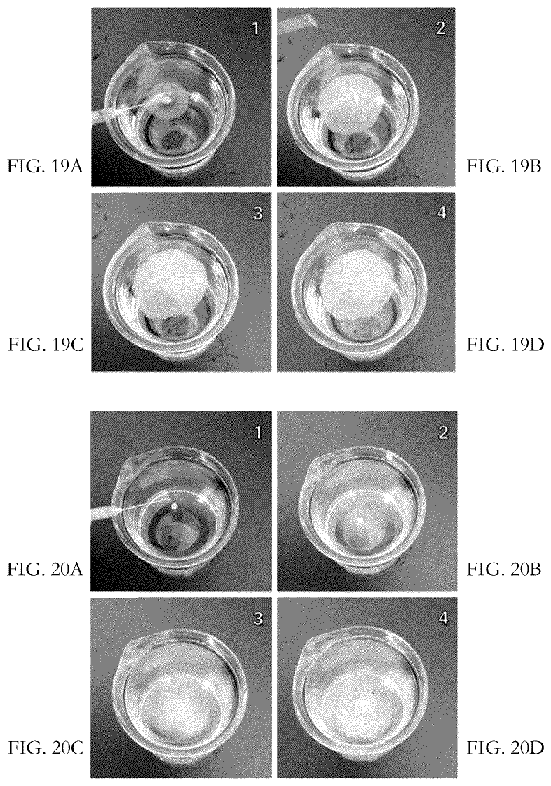

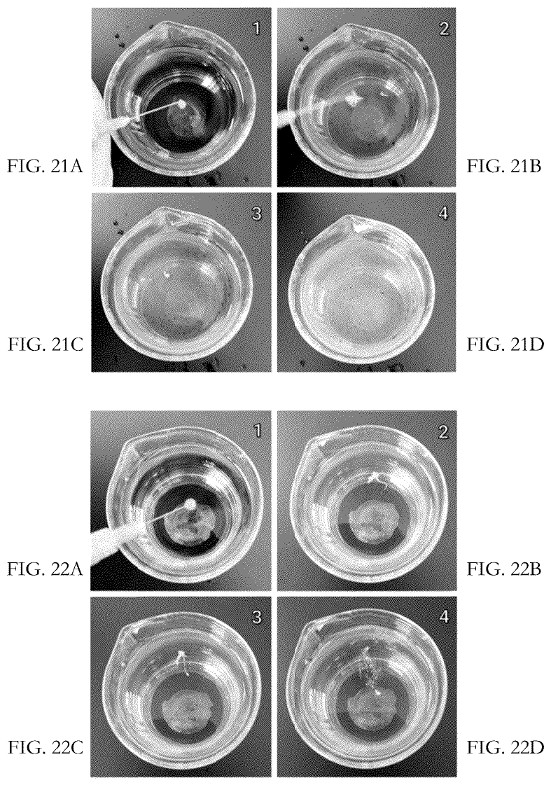

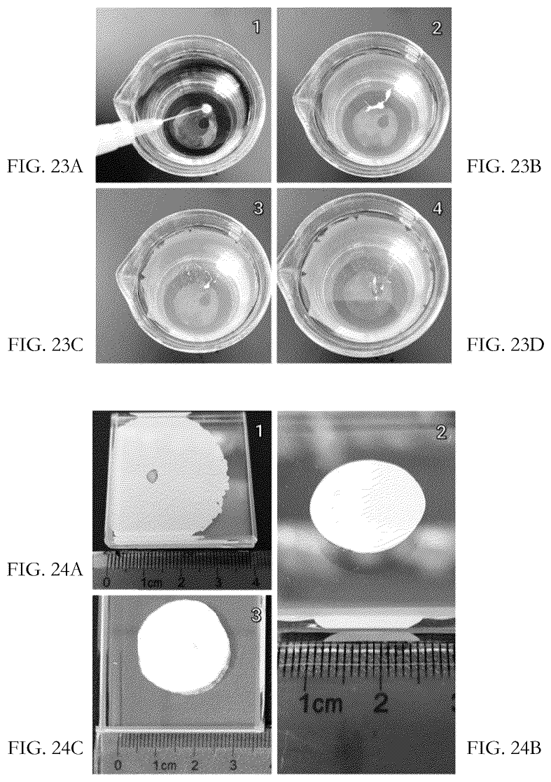

S1: Prepare 23 150 mL beakers with similar diameters, fill them with water, and inject one drop (approximately 0.01 g) of each of the 23 pastes (A, B, C, D, E, F, G, H, A1-A15) into each beaker through a 25 G root canal irrigation needle (except G and H). Record videos and observe the phenomena, capturing 4 images (A to D) from each video as a flow animation, as shown in FIGS. 1A-23D.

S2: Observe the color change immediately after preparation.

### 3. Test Results

The test results are shown in Table 2:

TABLE 2
Water Dispersion Results

| Image | | | | | | |
|---|---|---|---|---|---|---|
| and Paste | | | Sinks in | Water | Dispersion | Color |
| Code | Surfactant Type | | Water | Dispersion | Time (s) | Change |
| A | None | | ∘∘∘ | ++ | 2 | No Change |
| B | None | | ∘∘∘ | ++ | 2 | Fades |
| C | None | | ∘ | + | No | No Change |
| | | | | | Dispersion | |
| D | None | | ∘∘ | ++ | 1 | No Change |
| E | Added RH60 | | ∘∘∘ | ++++ | 2 | No Change |
| F | Added RH60 | | ∘∘∘ | ++++ | 4 | Fades |
| G | — | | ∘ | + | No | — |
| | | | | | Dispersion | |
| H | — | | ∘∘ | ++ | Local | — |
| | | | | | Dispersion | |
| A1 | Cationic | PHMB | ∘∘∘ | ++++ | 2 | Fades |
| A2 | | PHMG | ∘∘∘ | ++ | 3 | Fades |
| A3 | | BZK | ∘∘ | ++++ | 5 | No Change |
| A4 | | DDAC | ∘∘ | ++ | 8 | No Change |
| A5 | | CHX | ∘∘∘ | ++++ | 2 | No Change |
| A6 | | 123 | ∘∘∘ | ++++ | 19 | No Change |
| A7 | Anionic | SMG | ∘∘ | ++ | 4 | No Change |
| A8 | | SDS | ∘∘ | +++ | 14 | No Change |
| A9 | Nonionic | APG-10 | ∘∘∘ | +++ | 11 | No Change |
| A10 | | AEO-9 | ∘ | + | No | No Change |
| | | | | | Dispersion | |
| A11 | | TW-80 | ∘∘∘ | +++ | 3 | No Change |
| A12 | | F127 | ∘∘∘ | ++++ | 12 | No Change |
| A13 | | RH60 | ∘∘∘ | ++++ | 1 | No Change |
| A14 | Amphoteric | OA12/14 | ∘∘ | ++ | 11 | No Change |
| A15 | | BS-12 | ∘∘ | +++ | 11 | No Change |

Notes:
\textcircled{1}. “∘∘∘” means no paste sinks in the water during dispersion and can disperse on the water surface; “∘∘” means some paste sinks in the water during dispersion; “∘” means cannot disperse on the water surface, all paste sinks in the water.
\textcircled{2}. “++++” means evenly disperses and distributes over the entire water surface; “+++” means can disperse and distribute evenly on part of the water surface; “++” means limited dispersion, cannot distribute evenly on the water surface; “+” means cannot disperse in water.
\textcircled{3}. Color change indicates unstable formulation.
\textcircled{4}. Overall evaluation standard: the more and “+”, the shorter the “dispersion time”, and “no color change” indicate the optimal formulation.

### 4. Test Conclusions

4.1. By comparing pastes A, B, and C, it can be concluded that the difference lies in the solvents. Paste C using water as the solvent shows no dispersion in water. Paste B using propylene glycol and paste A using polyethylene glycol 200 show short dispersion times and no sinking into water but can only unevenly and limitedly disperse on the water surface, with paste B showing fading. Therefore, polyethylene glycol is the most suitable solvent, while water or other solvents may lead to poor dispersion or instability.

4.2. Comparing paste D with paste A, the difference is in the matrix. Paste D using hydroxypropyl methylcellulose shows more sinking than paste A using polyvinylpyrrolidone. Hence, polyvinylpyrrolidone is a better matrix system.

4.3. From Table 2, it can be seen that different surfactants have varying effects on the dispersion of the paste in water. Cationic surfactants generally improve dispersion and shorten dispersion time, but only some types show optimal characteristics without sinking, high dispersion, quick dispersion, and stability. Some cationic surfactants cause fading. CHX shows the best performance. Anionic surfactants result in sinking, poor dispersion, and slow dispersion, making them unsuitable. Nonionic surfactants improve dispersion overall, but only some types show optimal characteristics without sinking and high dispersion. AEO-9 shows negative effects, and RH60 shows the best performance. Amphoteric surfactants result in sinking, poor dispersion, and slow dispersion, making them unsuitable.

### III. Comparison of Flowability with Existing Products

### 1. Test Materials

Formula 3 paste from Table 3, G: Commercially available domestic calcium hydroxide paste (LQC), H: Commercially available imported calcium hydroxide paste (APC)

### 2. Test Method

The test was conducted according to ISO6876 standards.

### 3. Test Results

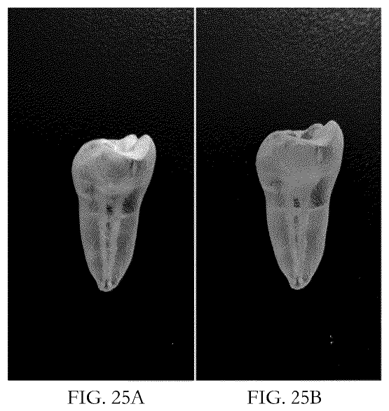

As shown in FIGS. 24A to 24C, the average diameter of the pressed film for Formula 3 paste was 40.0 mm (measured six times in different directions and averaged), the diameter for LQC was 15.3 mm (measured six times in different directions and averaged), and the average diameter for APC was 20.5 mm (measured six times in different directions and averaged).

Therefore, it can be concluded that the flowability of the paste of the present disclosure is significantly superior to that of existing related products, exhibiting high flowability characteristics.

As shown in FIGS. 25A to 25B, FIGS. 25A to 25B provide a comparative photograph of the state of the root canal before and after being flushed with 3 mL of water after injecting Formula 3 into a 3D-printed tooth root canal. FIG. 25A shows the state of the root canal before flushing, and FIG. 25B shows the state after flushing. By comparing the images, it can be observed that the root canal was very clean after flushing, with no residual Formula 3 paste.

### IV. Specific Embodiments

### Example 1

The formulation is prepared with the following raw materials in mass percentages: 10-60% calcium hydroxide, 15-35% zirconium oxide, 0.1-5.0% polyoxyethylene (60) hydrogenated castor oil, 0.5-5.0% polyvinylpyrrolidone, and the remainder is polyethylene glycol 200.

### Preparation Method:

1. According to the mass ratio, take the above raw materials. First, fully swell the polyvinylpyrrolidone in polyethylene glycol 200 to obtain a homogeneous solution A with no more than 20% polyvinylpyrrolidone/polyethylene glycol 200.

2. Add calcium hydroxide, zirconium oxide, and polyoxyethylene (60) hydrogenated castor oil to homogeneous solution A and fully homogenize and disperse using a homogenizer to obtain the final product.

### Example 2

Table 3 lists the raw materials and their mass ratios for 8 formulations. For each formulation, the raw materials are taken according to the mass ratios and a homogeneous paste is prepared according to the preparation method described in Example 1.

TABLE 3
Raw Materials and Their Mass Percentage Ratios

| | Ca(OH)₂ | ZrO₂ | RH60 | PVP | PEG-200 |
|---|---|---|---|---|---|
| Formulation 1 | 15.0 | 35.0 | 0.5 | 1.0 | 48.5 |
| Formulation 2 | 20.0 | 30.0 | 0.5 | 1.0 | 48.5 |
| Formulation 3 | 25.0 | 27.0 | 0.5 | 1.0 | 46.5 |
| Formulation 4 | 30.0 | 25.0 | 0.5 | 1.0 | 43.5 |
| Formulation 5 | 15.0 | 35.0 | 1.0 | 0.5 | 48.5 |
| Formulation 6 | 20.0 | 30.0 | 1.5 | 1.5 | 47.0 |
| Formulation 7 | 25.0 | 27.0 | 2.0 | 2.0 | 44.0 |
| Formulation 8 | 30.0 | 25.0 | 0.5 | 0.5 | 44.0 |
| Formulation 9 | 60.0 | — | 5.0 | 3.0 | 32.0 |
| Formulation 10 | 10.0 | — | 0.1 | 5.0 | 84.9 |
| Formulation 11 | 35.0 | 15.0 | 3.0 | 3.0 | 44.0 |
| Formulation 12 | 35.0 | 15.0 | 1.5 | 2.0 | 46.5 |

In Table 3, RH60: Polyethylene Glycol (60) Hydrogenated Castor Oil; PVP: Polyvinylpyrrolidone; PEG-200: Polyethylene Glycol 200.

In the formulation, PEG-200 can be replaced by PEG-400 or PEG-600. RH60 can be replaced by other polyethylene glycol hydrogenated castor oils with different molecular weights, such as Polyethylene Glycol (40) Hydrogenated Castor Oil.

The above descriptions are merely preferred embodiments of the present disclosure and are not intended to limit the disclosure. Any modifications, equivalent replacements, or improvements made within the spirit and principles of the present disclosure should be included within the scope of the protection of the present disclosure.

1-10. (canceled)

11. A matrix of an anhydrous root canal disinfectant, wherein the matrix comprises, based on the total mass percentage of the anhydrous root canal disinfectant, following components: 0.5-5.0% polyvinylpyrrolidone, 0.1-5.0% nonionic or cationic surfactant, and 32.0-85.0% polyethylene glycol with a molecular weight of 200-600.

12. An anhydrous root canal disinfectant comprising the matrix according to claim 11 and a hydroxide of alkaline earth metal, wherein the anhydrous root canal disinfectant is a paste.

13. The anhydrous root canal disinfectant according to claim 12, wherein the hydroxide of alkaline earth metal is calcium hydroxide.

14. The anhydrous root canal disinfectant according to 12, wherein the nonionic surfactant is polyoxyethylene ether hydrogenated castor oil, or the cationic surfactant is chlorhexidine.

15. The anhydrous root canal disinfectant according to claim 12, wherein the amount of the hydroxide of alkaline earth metal is 10-60% of the total mass of the anhydrous root canal disinfectant.

16. The anhydrous root canal disinfectant according to claim 12, further comprising an X-ray radiopaque agent, which is zirconium oxide.

17. The anhydrous root canal disinfectant according to claim 12, further comprising an antimicrobial agent, wherein the antimicrobial agent is single-chain and/or double-chain cationic quaternary ammonium salt and/or chlorhexidine.

18. The anhydrous root canal disinfectant according to claim 12, wherein comprising 0.5-5.0% polyvinylpyrrolidone, 0.1-5.0% polyoxyethylene ether hydrogenated castor oil, 10-60% calcium hydroxide, 15-35% zirconium oxide, and the remainder is polyethylene glycol with a molecular weight of 200-600 and other auxiliaries.

19. A method for preparing the anhydrous root canal disinfectant according to claim 12, comprising following steps:
fully swelling polyvinylpyrrolidone in polyethylene glycol to obtain a homogeneous solution A; and
adding the remaining components to the homogeneous solution A, and homogenizing and dispersing to obtain the final product.

20. A dental root canal treatment method using the anhydrous root canal disinfectant according to claim 12, comprising:
injecting the anhydrous root canal disinfectant into a root canal through a root canal irrigation needle for disinfection; and
rinsing the anhydrous root canal disinfectant with water or a root canal irrigation solution.

21. The matrix of an anhydrous root canal disinfectant according to 11, wherein the nonionic surfactant is polyoxyethylene ether hydrogenated castor oil.

22. The matrix of an anhydrous root canal disinfectant according to 11, wherein the nonionic surfactant is polyethylene glycol (40) hydrogenated castor oil or polyoxyethylene (60) hydrogenated castor oil.

23. The matrix of an anhydrous root canal disinfectant according to 11, wherein the cationic surfactant is chlorhexidine.

24. The anhydrous root canal disinfectant according to claim 12, wherein the amount of the hydroxide of alkaline earth metal is 35-60% of the total mass of the anhydrous root canal disinfectant.

25. The anhydrous root canal disinfectant according to claim 12, not comprising water or glycerol.
